(19) United States
(12) Patent Application Publication
(10) Pub. No.: US 2006/0008541 A1
(43) Pub. Date: Jan. 12, 2006

(54) Sodium channel agonist

(75) Inventor: Won-Kyung Ho, Seoul (KR)

Correspondence Address: JHK LAW, P.O. BOX 1078, LA CANADA, CA, 91012-1078, US

(73) Assignee: BIZMEDIC CO., LTD.

(21) Appl. No.: 11/177,260

(22) Filed: Jul. 8, 2005

Related U.S. Application Data
(60) Provisional application No. 60/586,601, filed on Jul. 8, 2004.
(60) Provisional application No. 60/586,680, filed on Jul. 8, 2004.

(51) Int. Cl. A61K 36/537

(52) U.S. Cl. 424/746

(57) ABSTRACT

The present application discloses a method of slowing inactivation of sodium current in a cell by contacting the cell with dimethyl lithospermate B.

### CROSS-REFERENCE To RELATED APPLICATIONS

The present application claims priority to U.S. Provisional Application No. 60/586,601, filed Jul. 8, 2004, and U.S. Provisional Application No, 60/586,680, filed Jul. 8, 2004, the contents of which are incorporated hereby in their entirety.

### BACKGROUND OF THE INVENTION

1. Field of the Invention

The present invention relates to an agonist specific for sodium channel. The invention also relates to methods of treating various impairments in cardiac rhythm and contractility. The invention also relates to an herbal composition containing the agonist.

2. General Background and State of the Art

The voltage-gated Na⁺ channel (VGSC) is the major determinant underlying the upstroke phase of the action potential in most excitable cells. The gating of VGSC is modulated by various intracellular signal transduction mechanisms and by drugs. In addition, mutations of VGSC are known to be responsible for a variety of conditions, such as cardiac arrhythmia and epilepsy. Long QT-3 and Brugada syndromes are well characterized examples of gain-of-function and loss-of-function mutations of VGSC, respectively (Grant et al., 2002, Veldkamp et al., 2000).

Drugs targeting VGSCs are widely used clinically as local anaesthetics, muscle relaxants, and anti-arrhythmic and antiepileptic agents. They are commonly Na⁺ channel inhibitors. Recently, synthetic Na⁺ channel agonists have been proposed as a possible new pharmacological tool for improving cardiac contractility in congestive heart failure patients (Muller-Ehmsen et al., 1998). These agents are characterized by the slowing of the inactivation phase of the Na⁺ current (I_{Na}) and the prolongation of action potential duration (APD). These effects are expected to increase Na⁺ influx and intracellular Na⁺ load, which in turn leads to a positive inotropic effect mediated by the Na⁺/Ca²⁺ exchange activities of ventricular myocytes. Investigations of the therapeutic potential of Na⁺ channel agonists for the treatment of Brugada syndrome may also be interesting, particularly because no therapeutic agent is yet available for this life-threatening disease. However, synthetic Na⁺ channel agonists have a critical drawback to the clinical application because of the pro-arrhythmic effect. Most synthetic Na⁺ channel agonists induce a slowing of I_{Na} inactivation and develop a persistent Na⁺ current (Yuill et al., 2000), causing the generation of early after-depolarisation (EAD; Ruegg & Nuesch, 1995; Yuill et al., 2000). Such pro-arrhythmic risk makes these drugs less suitable for clinical application.

A natural substance is identified herein from the root extract of Salvia miltiorrhiza (Labiatae), which functionally resembles synthetic Na⁺ channel agonists. The root of this plant, known as “Danshen”, is used in oriental medicine to improve blood circulation. It has been reported to contain lithospermate B (LSB) as a major active constituent (Tanaka et al., 1989), which reportedly enhances endothelium-dependent vasodilatation (Kamata et al., 1993), and shows beneficial effects on renal injury (Lee et al., 2003). The effects of the root extract of S. miltiorrhiza on cardiac action potential (AP) was examined, and found that it increases the AP duration in isolated rat ventricular myocytes. When the active component of the extract was purified by repeated activity-guided fractionation, it was not LSB, but dimethyl LSB (dmLSB), which was present as a minor component (FIG. 1). Electrophysiological investigation confirmed the identity of the target channel of dmLSB.

### SUMMARY OF THE INVENTION

In one aspect, the invention is directed to a method of slowing inactivation of sodium current in a cell, which method comprises contacting the cell with dimethyl lithospermate B. The invention is also directed to treating a person or animal with arrhythmia or Brugada syndrome or generally a person or animal with impaired cardiac rhythm or contractility by administering a composition containing dimethyl lithospermate B to a person in need thereof. The person being treated may check for the effects of the treatment by usual cardiac measurement techniques. Dimethyl lithospermate B may be administered to the patient in many different forms such as by injection of a pharmacologically acceptable composition containing the dimethyl lithospermate B.

Herbal compositions containing dimethyl lithospermate B are also contemplated. An alcohol extract of the Salvia plant contains dimethyl lithospermate B. And therefore, a concentrated version of the Salvia plant may be used as the dimethyl lithospermate B carrier to the person in need of dimethyl lithospermate B. In one aspect, dimethyl lithospermate B may be administered through a drink or formulated into a tablet.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows chemical structure of dimethyl-lithospermate B.

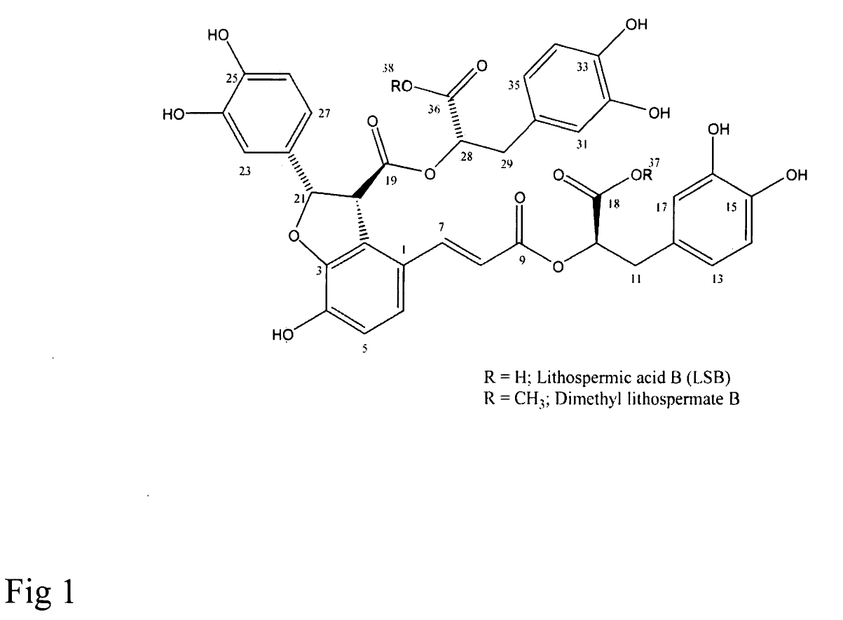

FIG. 2 shows effects of dmLSB on cardiac action potential. A. Change in AP duration during the bath application of dmLSB. All data points were obtained from the same cell. Horizontal bars above the plot indicate the duration of the bath application of dmLSB. APs were evoked by depolarising current pulse (55 pA, 5 ms, 2 Hz). APD₉₀ (AP duration at 90% repolarisation) was obtained from the average of five sequential APs selected every 12 seconds. B. Left, Exemplary AP records at various concentration of dmLSB (a: control, b: 4 μM, c: 10 μM, d: 20 μM, e: wash-out of dmLSB). The time point of each AP record was indicated by a triangle in A. Right, APD₉₀ as a function of the concentration of dmLSB. APD values obtained from seven different cells were superimposed (smaller open symbols). At each concentration of dmLSB, a mean value for APD₉₀ was also superimposed (larger closed circles; error bars, SEM). Asterisks mean statistical significance (paired t-test; p<0.01).

FIG. 3 shows effects of dmLSB on the whole-cell current A. Current responses to step voltage pulses (inset, −120 mV to +80 mV) from Vₕ of −80 mV were recorded in whole-cell mode using K⁺-rich pipette solution. Left: control condition, Right: in the presence of 10 μM dmLSB. B. Voltage-dependence of current amplitude measured at 20 ms (left) and at 950 ms (right) of the step pulses. The I-V relationships before (open circle) and after (closed circle) the dmLSB application were superimposed in each plot.

FIG. 4 shows effects of dmLSB on cardiac inward current A. Current in response to the double step depolarising pulses (inset). Sets of current traces before (left) and after (middle) the application of 10 μM dmLSB. Notice the slowing of the fast inward current in the presence of dmLSB (arrow heads). Right, voltage-dependence of peak amplitude of the current evoked by the second step pulses. The I-V relationships before (open circle) and after (closed circle) the dmLSB application were superimposed (n=4; error bars, SEM). B. Effects of dmLSB on the inward current in the presence of 100 μM tetrodotoxin (TTX). Sets of current traces in response to the depolarisation pulses (inset) before and after bath-applying dmLSB (10 μM).

FIG. 5 shows effects of dmLSB on cardiac I_{Na} in the condition of low [Na⁺] A. Whole-cell I_{Na} was elicited from a Vₕ of −120 mV to test potentials ranging from −80 to +10 mV in 10 mV step increments before (left) and after (right) application of 10 μM dmLSB to the ventricular myocyte from the young rat (3 weeks old). B. A pair of I_{Na} were superimposed in control (grey line) and in dmLSB (black line) at the same test potential for comparison. Inset, the same I_{Na} at −20 mV in the expanded vertical scale. Notice the absence of persistent I_{Na} in dmLSB. C. Left shows the I-V relationships, which represent the peak I_{Na} values before (open circle) and after (closed circle) the application of dmLSB. Right, the activation curves were estimated from I-V curves of four different cells. The half activation voltage was significantly shifted to the positive direction by dmLSB (n=4, paired t-test p<0.05). D. Left, time constants estimated from fitting inactivation phases with mono- or bi-exponential function. Only a single time constant for each test potential was plotted when mono-exponential function was sufficient for fitting (for V_{T}=−60, −50 and −40 mV). Right, the plot of relative amplitude of slow component induced by dmLSB as a function of test potential.

FIG. 6 shows no effect of dmLSB on the steady-state inactivation of I_{Na}. A. I_{Na} elicited by step pulses to −20 mV after 500 ms prepulses of various levels in control condition (left) and in the presence of 20 μM dmLSB (right). B. Summary of mean availability of Na⁺ channel as a function of pre-pulse voltages in the absence (open circle, n=28) and in the presence (closed circle, n=32) of 20 μM dmLSB. Data were fitted by 1−(1/(1+exp((V_{1/2}−Vₘ)/k))). V_{1/2} and k were −75.7 and 10.20 for control, and −77.8 and 9.99 for dmLSB, respectively. Error bars, SEM.

FIG. 7 shows dose-response relationship. A. upper, The change in the relative amplitude of slowly inactivating Na⁺ current (I_{Slow})to the peak I_{Na} as a function of whole-cell recording time. A pair of step pulse to −20 mV from the prepulse of −130 mV and from that of −40 mV were applied every 10 s to obtain I_{Na}. The dmLSB-induced I_{Slow} was obtained by subtracting I_{Na} in control condition from I_{Na} in the presence of dmLSB. Lower, I_{Na} (grey) traces in control condition and in the presence of dmLSB and I_{Slow} (black) were superimposed. The time when each set of current records was obtained is marked in the upper plot with ‘a’ and ‘b’. B. Relative amplitude of slow components induced by dmLSB to the peak I_{Na} was plotted as a function of the concentration of dmLSB. The relationship was fitted to a Hill equation. The half-maximum effective concentration and Hill coefficient were 21.1 μM and 1.02, respectively.

FIG. 8 shows effects of intra- and extra-cellular dmLSB on I_{Na}. The whole-cell mode was obtained with the pipette solution containing 20 μM dmLSB. I_{Na} was evoked by test pulse to −20 mV from Vₕ of −80 mV. A. The time course of peak I_{Slow} (I_{Na, dmLSB}−I_{Na, control}) during bath application of 5 μM dmLSB. The arrow heads indicate the start and the end of the solution changes. B. Current traces obtained from before (left) and after (middle) the bath-application of dmLSB to the same cell. Extra-cellular dmLSB slowed the I_{Na} inactivation. The dmLSB effect was completely reversed after the wash-out of external dmLSB (right). In each plot, the initial I_{Na} recorded just after break-in was superimposed for comparison (grey traces).

FIG. 9 shows a table comparing the I_{Na} decay rate, AP duration and steady state (S-S) inactivation of the various derivatives of LSB, indicating that I_{Na} slowing without causing early afterdepolarization (EAD) is the unique nature of dmLSB. EAD is an indication of arrhythmogenic potential.

### DETAILED DESCRIPTION OF THE PREFERRED EMBODIMENTS

In the present application, “a” and “an” are used to refer to both single and a plurality of objects.

Voltage-gated Na⁺ channel blockers have been widely used as local anaesthetics and anti-arrhythmic agents. It has recently been proposed that Na⁺ channel agonists can be used as inotropic agents. Here we report the identification of a natural substance that acts as a Na⁺ channel agonist.

Using the patch clamp technique in isolated rat ventricular myocytes, we investigated the electrophysiological effects of the substances isolated from the root extract of Salvia milliorrhiza, which is known as “Danshen” in Asian traditional medicine. By the intensive activity-guided fractionation, dimethyl lithospermate B (dmLSB) was identified as the most active component, while LSB, which is the major component of the extract, showed negligible electrophysiological effect. Action potential duration (APD₉₀) was increased by 20 μM dmLSB from 58.8±12.1 ms to 202.3±9.5 ms. In spite of the prolonged APD, no early after-depolarisation was observed.

dmLSB had no noticeable effect on K⁺ or Ca²⁺ currents, but selectively affected Na⁺ currents. dmLSB slowed the inactivation kinetics of Na⁺ currents by increasing the proportion of slowly inactivating component without inducing any persistent Na⁺ current. The relative amplitude of slow component compared to the peak fast Na⁺ current was increased dose-dependently by dmLSB (EC₅₀=20 μM). Voltage-dependence of inactivation was not affected by dmLSB, while voltage-dependence of activation shifted by 5 mV to the depolarised direction.

Since the APD prolongation by dmLSB did not provoke EAD, which is thought as a possible mechanism for the proarrhythmia seen in other Na⁺ channel agonists, dmLSB is indicated to be an excellent candidate for a Na⁺ channel agonist.

Lithospermate B (LSB) is a caffeic acid tetramer, and was originally isolated from the root of Salvia miltiorrhiza (also called as dansham or danshen), an important herb in oriental medicine, which has been used to treat cardiovascular disease. LSB, the main component of S. miltiorrhiza, has been shown to induce endothelium-dependent vasodilation, which was inhibited by NG-monomethyl-L-arginine (Kamata et al., 1993), and to inhibit the norepinephrine-induced contraction of the aortic strips by reducing Ca²⁺ mobilization (Nagai et al., 1996). The effect of LSB on the heart has also been reported. LSB was found to reduce the myocardial damage induced in a rabbit ischemia-reperfusion model (Fung et al., 1993). However, no report has been issued on the effects of LSB on cardiac electrical activity. It has now been discovered that LSB itself has no effect on cardiac action potential, but dimethyl ester form of LSB (dmLSB) increases the APD of the rat ventricular myocytes. A voltage clamp study revealed that the slowing of I_{Na} inactivation underlies the APD prolongation, and that no current system except fast I_{Na} was affected by dmLSB. The prolongation of APD induced by dmLSB was not associated with the generation of EAD. Consistent with the absence of the EAD generation, dmLSB developed no persistent I_{Na}. These pharmacological properties of dmLSB indicates usefulness in clinical application.

Comparison of the Effect of dmLSB on I_{Na} with Other Lipid-soluble Na-channel Agonists

The gating parameters of Na⁺ channels are highly susceptible to a number of lipid-soluble alkaloids, insecticides, and toxins from tropical frog. In spite of their structural diversity, these lipid-soluble toxins share a common mechanism of action, which suggests the presence of a receptor in a strongly hydrophobic region of Na⁺ channel. The most thoroughly studied compounds are veratridine, bartrachotoxin, aconitine and pyrethroids (Caterall, 1980; Narahashi, 1996; Wang & Wang, 2003). Lipid-soluble alkaloids (also called as Na⁺ channel agonist) shift the voltage dependence of activation to more negative potentials and inhibit or completely block inactivation. These two effects combine to cause persistent activation of a fraction of Na⁺ channels at resting Vₘ.

A recent study reported that pyrethroids also increase the window region of cardiac I_{Na}: a potential range over which activation and inactivation curves overlap (Spencer et al., 2001). Moreover, type II pyrethroids, fenpropathrin, produced leftward shifts in both the activation and the inactivation curves of I_{Na}, resulting the shift of window region to a more negative potential. These changes might be involved in the generation of EADs on a prolonged plateau phase in cardiac ventricular myocytes (Spencer et al., 2001).

The effect of dmLSB on I_{Na} is distinct from that of other Na⁺ channel agonists in that it shifts the activation curve to the positive direction and has no effect on the steady-state inactivation curve of I_{Na}. In addition, while dmLSB slowed down the inactivation kinetics of I_{Na}, it produced no sustained component of I_{Na}. These characteristics may be related to the change of APD caused by dmLSB. It prolonged the APD without provoking EADs. Considering that generation of EAD is responsible for generation of arrhythmias by these alkaloids, the characteristics of the dmLSB effect are of particular interest. It remains to be elucidated whether the lack of EAD in the presence of dmLSB indicates that dmLSB does not have arrhythmogenic potentials.

Clinical use of dmLSB: Comparison of the Effect of dmLSB on I_{Na} with Other Inotropic Agents

DPI 201-106, BDF 9148, BDF 9198, and its related compounds have been reported to exert a positive inotropic effect on cardiac myocardium (Muller-Ehmsen et al., 1998). Subsequent studies revealed that the slowing of I_{Na} inactivation is responsible for the inotropic effects (Yuill et al., 2000). In addition to the slowing of I_{Na} inactivation, these drugs were found to induce a persistent I_{Na}, causing the increase in net depolarising current during action potential. Accordingly, they induced extensive prolongation of APD and of the QT-interval in ECG, which is associated with risk of pro-arrhythmia mediated by EAD (Ruegg & Nuesch, 1995; Yuill et al., 2000). Such pro-arrhythmic risk makes these drugs less suitable for clinical application. Moreover, the effects of DPI 201-106 and BDF 9148 are not restricted to Na⁺ channels, but they inhibit the L-type calcium current and inward rectifier K⁺ current (Raven et al., 1995).

Recently, it was reported that KB130015, a newly developed amiodarone derivative, slowed I_{Na} inactivation without developing persistent I_{Na} (Macianskiene et al., 2003). From these properties, KB130015 is expected to be less pro-arrhythmic than other synthetic Na⁺ channel modulators. However, it reduces I_{Ca} and consequently reduces APD, making it less useful as an inotropic agent (Macianskiene et al., 2003). In contrast, dmLSB did not affect any current system in cardiac myocytes, while its effect on the inactivation kinetics of cardiac I_{Na} was similar to those of KB130015.

Brugada syndrome is a genetic disorder in which mutations are identified in SCN5A, the gene encoding cardiac Na⁺ channel, Naᵥ1.5. Multiple mutations have been identified in virtually all regions of Naᵥ1.5, and it has been shown that many of the Brugada syndrome mutations alter Na⁺ channel's gating function in a manner that reduces the Na⁺ channel availability. Delayed recovery of the Na⁺ channel from slow inactivation state has been proposed as the main functional defect found in Brugada syndrome (Wang et al., 2000). A reduced Na⁺ channel function causes the loss of AP dome, preferentially in epicardial cells, which leads to early epicardial repolarization. The consequent heterogeneity of repolarization in the right ventricular wall increases the risk of reentrant arrhythmia. Prolongation of APD by Na⁺ channel enhancement is a plausible therapeutic strategy for the rectification of APD shortening found in Brugada syndrome.

Therapeutic Composition

The inventive therapeutic compound may be administered to human patients who are either suffering from, or prone to suffer from heart disease such as arrhythmia or Brugada syndrome by providing compounds that is an agonist to sodium channel. In particular, the compound is dimethyl lithospermate B.

The formulation of therapeutic compounds is generally known in the art and reference can conveniently be made to Remington's Pharmaceutical Sciences, 17th ed., Mack Publishing Co., Easton, Pa., USA. For example, from about 0.05 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, intradermal or suppository routes or implanting (e.g., using slow release molecules by the intraperitoneal route. Depending on the route of administration, the compound may be required to be coated in a material to protect it from the action of enzymes, acids and other natural conditions which may inactivate the ingredient.

The compound may be administered in an adjuvant, co-administered with enzyme inhibitors or in liposomes. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water CGF emulsions as well as conventional liposomes.

The active compounds may also be administered parenterally or intraperitoneally. Dispersions can also be prepared in glycerol liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When the peptides are suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg/ml of carrier. In the case of compositions containing supplementary active ingredients, the dosages are determined by reference to the usual dose and manner of administration of the said ingredients.

Delivery Systems

Various delivery systems are known and can be used to administer the compound of the invention. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the heart system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

A composition is said to be “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

Herbal Formulation

dmLSB, which has been discovered to be a useful sodium channel agonist such that it can be used to treat a variety of heart conditions such as arrhythmia and Brugada syndrome or strengthen the heart in general may be formulated into an herbal composition, which may be eaten or imbibed through a tea formulation. The herbal composition may be mixed with a pre-calculated desired amount of dmLSB as a dietary supplement.

The herbal composition of the invention may include a single extract from any one of a plant belonging to the genus Salvia, or the composition may include a combination of a plurality of the Salvia plants. In one embodiment, the composition may comprise Salvia miltiorrhiza extract, in particular, an extract that is enriched for dmLSB.

With regard to the formulation of dmLSB, if desired, the pharmaceutical composition to be administered may also contain minor amounts of non-toxic auxiliary substances such as wetting or emulsifying agents, pH buffering agents and the like, such as for example, sodium acetate, sorbitan monolaurate, triethanolamine oleate, and so on.

The amount of the herbal medicine in a formulation can vary within the full range employed by those skilled in the art, e.g., from about 0.01 weight percent (wt %) to about 99.99 wt % of the medicine based on the total formulation and about 0.01 wt % to 99.99 wt % excipient.

It is understood that by “pharmaceutical composition” or “herbal medicinal composition”, it is meant that the herbal composition is formulated into a substance that is to be administered purposefully for treating or preventing mild to moderate heart condition such as arrhythmia or Bruguda syndrome in an individual.

In an embodiment of the invention, special extracts of Salvia containing dmLSB is administered to a person suffering from mild heart condition such as arrhythmia or Bruguda syndrome to treat symptoms of arrhythmia or Bruguda syndrome.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

### Example 1

### Isolation of Rat Ventricular Myocytes

Ventricular myocytes were isolated from the hearts of 3-week or 6 to 7- week-old Sprague-Dawley rats of either sex. Rats were anaesthetized with pentobarbitone sodium (i.p. 200 mg kg⁻¹). Isolated hearts were mounted on a Langendorff perfusion apparatus, washed at 37° C. for 5 min with a modified Tyrode solution containing (mM): 143 NaCl, 5.4 KCl, 5 HEPES, 0.5 MgCl₂, 0.5 NaH₂PO₄, 1.8 CaCl₂, 10 glucose, adjusted pH to 7.4 with NaOH, and then perfused with Ca²⁺-free Tyrode solution for 5 min. After the hearts had stopped beating, Ca²⁺-free Tyrode solution containing collagenase (0.5 mg ml⁻¹, Type II, Worthington) was perfused for 30 min. Finally, this enzyme containing solution was washed out for 5 min with a high K⁺, low Cl⁻KB solution containing (in mM): 70 K-glutamate, 55 KCl, 10 HEPES, 3 MgCl₂, 20 taurine, 20 KH₂PO₄, 0.5 K-EGTA adjusted pH to 7.3 with KOH. A portion of the left ventricle was then dissected out and gently stirred with a forceps in KB solution. The isolated ventricular myocytes were kept in KB solution at 5° C. before use.

### Example 2

### Electrophysiological Recordings

Patch pipettes (1˜2 MΩ when filled with experimental solutions) were pulled from borosilicate glass capillaries (Harvard Apparatus Ltd, UK). Conventional whole-cell patch clamp technique was used to record membrane current or voltage from single ventricular myocytes. In current-clamp mode, action potentials were evoked by a brief supra-threshold current pulse. In voltage clamp mode, access resistance was monitored throughout the experiment, and data were accepted only when the access resistance was kept below 6 MΩ. Filtered signals (10 KHz) from a patch clamp amplifier (Biologic RK 300, France), were fed into AD/DA converter (PCI-MIO-16E-4, National Instrument, USA), digitized at 20 KHz, and stored in PC for later analysis. The flow rate of the perfusion solution was 0.5˜1 ml min⁻¹. All electrophysiological experiments were performed at room temperature (23˜25° C.). The statistical results in the text and in figures were presented as means ±S.E.M. (n=number of cells studied). Statistical significance was accepted for p value <0.05.

### Example 3

### Solutions and Drugs

The K-rich pipette solution containing (in mM): 90 K-aspartate, 30 KCl, 2 MgCl₂, 5 Mg-ATP, 10 HEPES, 5 K-EGTA, 5 diTris-creatine phosphate, 2.5 Na₂-creatine phosphate was used. Cs-aspartate internal solution contained (in mM): 90 Cs-aspartate, 20 CsCl, 2 MgCl₂, 5 Mg-ATP, 10 HEPES, 2.5 Na₂-creatine phosphate, 10 tetraethyl-ammonium chloride (TEA-Cl), 5 Cs-EGTA with pH adjusted to 7.3 using CsOH. When Cs-aspartate internal solution was used in combination with normal Tyrode (NT) bathing solution, the holding current level was inward at −80 mV, probably due to K⁺ influx via inward rectifier K channels. To prevent this inward holding current, KCl in the NT solution was substituted with equimolar CsCl.

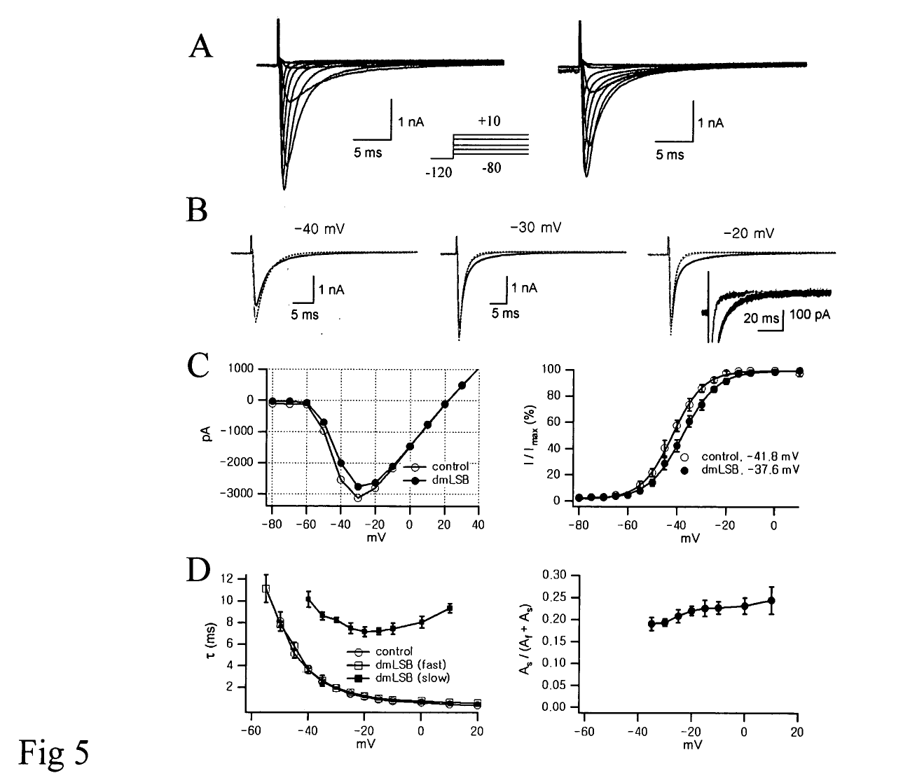

As sodium current (I_{Na}) in ventricular myocytes is so fast and large that membrane potential is prone to escape from the command voltage, it is necessary to reduce I_{Na} for a quantitative analysis. For this purpose, small ventricular myocytes isolated from young (3-weeks-old) rats were used, and currents recorded in a modified Cs⁺-based low Na⁺ bath solution (Yuill et al., 2000) which contained (in mM): 130 CsCl, 10 NaCl, 2.5 MgCl₂, 0.5 CaCl₂, 20 HEPES, 11 glucose, in experiments shown in FIG. 5. It was reported that the kinetics of I_{Na} in the standard solution (145 mM Na⁺) and in Cs-based solution (10 mM Na⁺) were similar.

To prepare a stock solution of dmLSB, it was dissolved in 50% ethanol at 40 mM, and stored at −20° C. Since the pharmacological effects of dmLSB decayed with time, stock solutions were used within 48 h of make-up. Tetrodotoxin (TTX) was purchased from Wako (Japan). Unless mentioned separately, all chemicals were purchased from Sigma-Aldrich Chemical Co. (St. Louis, USA).

### Example 4

### Isolation of Dimethyl Lithospermate B (dmLSB) from the Root Extract of S.miltiorrhiza

Dried roots of Salvia miltiorrhiza (6 kg) were soaked in MeOH for 7 days at room temperature. After filtration, the extract was concentrated under the reduced pressure to give 470 g of a dark syrupy MeOH extract. This was suspended in H₂O and sequentially partitioned with n-hexane, EtOAc, and BuOH. This process yielded 69 g in the n-hexane fraction, 52 g in the EtOAc fraction, 69 g in the BuOH fraction, and a water soluble residue. Upon the assessment of each part individually, only the EtOAc soluble part produced an effect of increasing AP duration in the isolated rat ventricular myocytes. Thus, a half of the EtOAc fraction (26 g) was subjected to octadecyl silica gel (ODS) column (φ6.0×60 cm) chromatography. The column was eluted in a stepwise gradient manner with 300 ml aliquots of MeOH in H₂O (0% to 100%), which delivered four fractions, i.e., Fr. 1 (3.2g), Fr. 2 (13g), Fr. 3 (2.4g), and Fr. 4 (7.0g). Among these fractions (Fr.1-Fr.4), Fr.2 was most potent, and was further purified by the Sephadex LH-20 column chromatography using 20% MeOH in CH₂Cl₂, which finally delivered 110 mg of dmLSB and 2.4 g of LSB. Moreover, LSB was easily converted to dmLSB by simple methylation of LSB in MeOH using p-toluenesulfonic acid as catalyt. The chemical structure of dmLSB was elucidated using ¹H-NMR and ¹³C-NMR data (Kohda et al, 1989).

### Example 5

### dmLSB Prolonged Action Potential in Rat Ventricular Myocytes

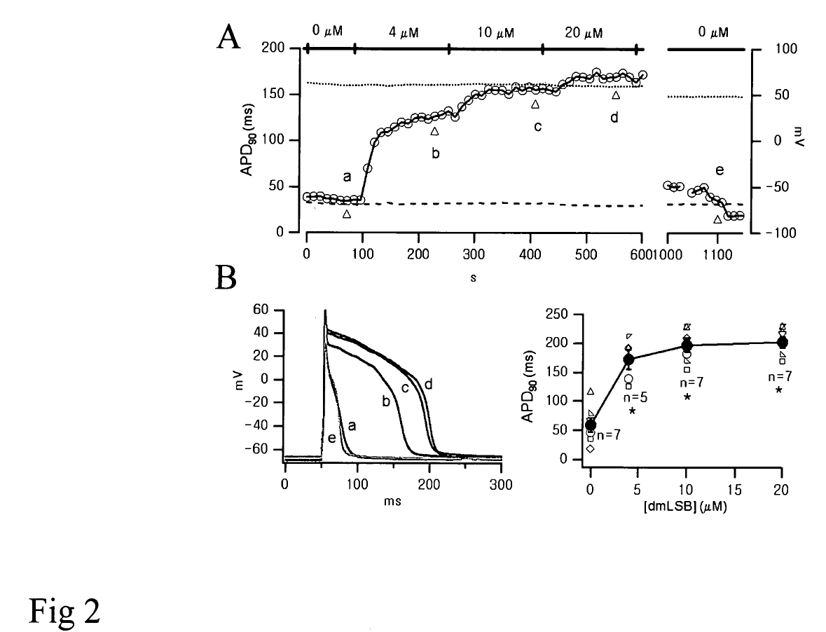

To investigate the effects of dmLSB on the electrical activity of the rat ventricular myocytes, changes were observed in AP shapes during the application of dmLSB to the bath solution. APs were evoked by applying 50-70 pA depolarising current pulses (5 ms in duration) every 500 ms through a patch pipette in current clamp mode with a whole-cell configuration. The effects of dmLSB on APD were quantified in terms of APD₉₀; defined as the APD measured at the voltage at which repolarization process is 90% complete. Time-profile of the change in APD₉₀ values averaging from five sequential APs selected every 12 seconds was plotted in FIG. 2A as a function of whole-cell recording time. Single APs recorded in control conditions and those recorded in the presence of dmLSB at various concentrations were superimposed in FIG. 2B (left). dmLSB markedly inhibited the initial rapid repolarization phase and prolonged the plateau phase. In contrast to the effect of other lipid-soluble Na⁺-channel agonists such as pyrethroids (Spencer et al., 2001), no secondary upward voltage deflection (referred to as early afterdepolarisation, EAD) was observed during the prolonged plateau phases. No changes either in resting membrane potential (RMP) or in the overshoot potentials of the APs were observed during the dmLSB superfusion (RMP: dashed line; overshoot potential: dotted line in FIG. 2A). The mean values for the RMP and the overshoot potential were −68.46±2.6 mV (n=7) and 53.53±8.8 mV (n=7), respectively. The steady-state APD₉₀ values obtained from seven myocytes at various concentrations of dmLSB are summarized in FIG. 2B (right). APD prolongation induced by dmLSB was maximal at 20 μM. At higher concentrations up to 100 μM, APD did not increase further, but slightly decreased (data not shown) with no EAD observed. The mean value of APD₉₀ under control condition was 58.8±12.1 ms and that in the presence of 20 μM dmLSB was 202.3±9.5 ms, indicating that dmLSB significantly prolonged APDs (paired t-test; n=7, p<0.01).

### Example 6

### dmLSB Affects the Initial Transient Component of the Current

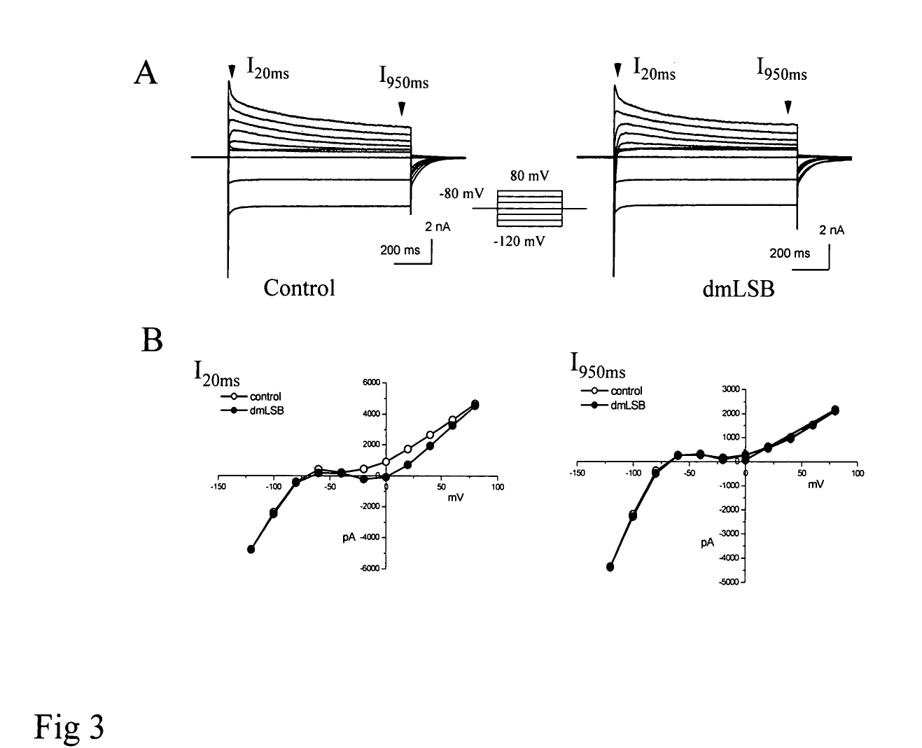

To elucidate the mechanism underlying APD change by dmLSB, voltage-clamp experiments were performed and the effects of dmLSB on the ionic currents in rat ventricular myocytes were investigated. Membrane currents were recorded in a voltage clamp mode using a patch pipette containing a K⁺-rich (140 mM) internal solution. FIG. 3A shows the current responses to a set of 1 s depolarisation pulses from a holding potential (Vₕ) of −80 mV to various voltages (inset) under control conditions and those in the presence of 10 μM dmLSB. Hyperpolarizing pulses induced large inward currents that showed a characteristic of inward rectifier K⁺ currents. Depolarising pulses induced complex current responses, each of which was probably composed of a fast inward Na⁺ current (I_{Na}), an L-type Ca²⁺ current (I_{Ca,L}), and a transient and a delayed outward K⁺ currents.

Steady-state amplitudes of the currents which were measured 950 ms after the start of the square pulses (I₉₅₀ₘₛ), showed no significant difference with or without 10 μM dmLSB, indicating that neither inward rectifier K⁺ currents nor delayed rectifier K⁺ currents were affected by dmLSB (FIG. 3B, right). However, initial amplitudes measured at 20 ms (I₂₀ₘₛ) in the presence of dmLSB differed from those measured under control conditions, and the I-V relationship deviated inwardly by dmLSB. The deviation was prominent in the voltage range from −20 mV to +20 mV, resulting in a bell-shaped current-voltage (I-V) relationship in dmLSB (FIG. 3B, left). The inward deviation of I-V relationship in the presence of dmLSB indicates that dmLSB might decrease a transient outward current or increase a transient inward current. However, this bell-shaped I-V relationship implies that the involvement of inward currents such as Ca²⁺ channels or Na⁺ channels is more likely.

### Example 7

### Enhancement of the Fast Inward Na+ Current by dmLSB

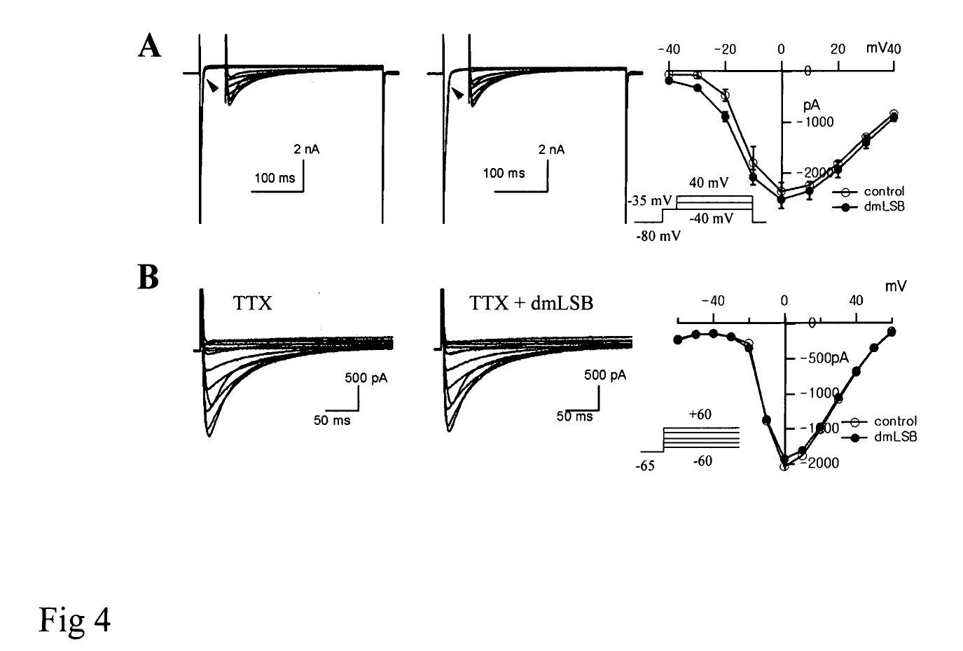

To examine the effect of dmLSB on inward currents, a Cs⁺-rich pipette solution containing 10 mM EGTA was used. To distinguish L-type Ca²⁺ currents (I_{Ca,L}) from Na⁺ currents (I_{Na}), a two-step pulse protocol was used (inset of FIG. 4A). From the holding potential of −80 mV, a ventricular myocyte was depolarised to −35 mV for 50 ms to inactivate I_{Na}, and this was followed by test pulses to various depolarisation to record I_{Ca,L}. The superimposed current traces under the control condition showed the rapid activation and inactivation of large I_{Na} at −35 mV followed by the slower activation and inactivation of I_{Ca,L} at test pulses (FIG. 4A). Although the peak amplitude of I_{Na} was unaffected by dmLSB, significant broadening of inactivation phase was observed. In contrast, I_{Ca,L} was hardly affected by dmLSB (10 μM).

The effect of dmLSB on I_{Ca,L} was further studied in the presence of 100 μM tetrodotoxin (TTX) to block I_{Na}. Depolarising test pulses (from −60 mV to +50 mV) for 200 ms from Vₕ of −65 mV were applied to activate I_{Ca,L}. FIG. 4B shows representative current traces before (left) and after (middle) dmLSB (10 μM) superfusion in the presence of TTX, from which the I-V curves of the peak amplitudes of I_{Ca,L} were obtained (FIG. 4B, right). No significant difference was observed in I-V curves between control and dmLSB. These results strongly indicate that dmLSB has little effect on I_{Ca,L}, but that it affects TTX-sensitive Na⁺ current.

### Example 8

### dmLSB Induces a Slow Component of I_{Na} Inactivation

To avoid the problem of voltage escape and to investigate the effects of dmLSB on I_{Na} in a quantitative manner, I_{Na} was recorded in low external [Na⁺] (10 mM). It is well known that the steady-state inactivation shifted spontaneously during whole-cell mode recording. To activate I_{Na} from the potential at which inactivation is fully recovered, depolarising test pulses were applied following a 500 ms pre-pulse to −120 mV. To eliminate the contamination of I_{Ca,L} and to isolate pure I_{Na}, current response to the same test pulse from holding potential (Vₕ) of −40 mV, which was regarded as I_{Ca,L}, was subtracted from that from Vₕ of −120 mV. The subtracted traces obtained in this way were not inhibited by 40 μM Ni²⁺, a T-type Ca²⁺ current blocker (I_{Ca,T}), indicating that I_{Ca,T} is not contaminated (Hagiwara et al, 1988).

FIG. 5A shows representative results of I_{Na} under control conditions and those in the presence of 10 μM dmLSB recorded from the same ventricular myocyte. For comparison, a pair of I_{Na} traces recorded before and after the application of dmLSB are superimposed at various test potentials (V_{T}) in FIG. 5B.

As was noticed in FIG. 4, the most prominent effect of dmLSB on I_{Na} was a slowing of the inactivation kinetics, which is well demonstrated in the superimposed current traces obtained at −20 mV (FIG. 5B, right). It is noted that the initial phase of fast inactivation was not affected, and the late phase was selectively slowed by dmLSB. Although dmLSB slowed the inactivation, it was completed within 50 ms without leaving a persistent inward current (inset; the same set of traces in an expanded scale), indicating that dmLSB does not enhance the persistent sodium current, but slows down the inactivation process of the fast I_{Na}.

It is noted that amplitude of I_{Na} decreased slightly by dmLSB. The voltage dependence of peak I_{Na} amplitude is further analysed in the I-V relationships (FIG. 5C). The reversal potential (Eᵣₑᵥ) was close to the expected equilibrium potential for Na⁺ (+20 mV). The peak amplitude of I_{Na} was reduced slightly by dmLSB in the hyperpolarized range below −20 mV, while they were similar in the more depolarised range where Na⁺ channels are fully activated. Accordingly, the activation curve that was obtained by dividing the current amplitude by the electromotive force, V-Eᵣₑᵥ, shifted to the right direction by 5 mV in the presence of dmLSB (FIG. 5C, right). The mean voltage for half-maximal activation in the presence of dmLSB was −37.28±0.8 (n=4), which is significantly more depolarised than that in control condition (−42.3 5±1.1; paired t-test, p<0.05, n=4).

For further investigation of the effect of dmLSB on the inactivation kinetics, the decay phase of I_{Na} was fitted with an exponential function. Under control conditions, a mono-exponential curve was sufficient to fit the decay phase of I_{Na} (dotted line in FIG. 5B). In the presence of dmLSB, however, the decay phase clearly deviated from the mono-exponential function, and a slow component emerged. The discrepancy became more pronounced as V_{T} was more depolarised. When the decay phase was fitted with a bi-exponential function, the amplitude of the slow component (A_{S}) was estimated to be 22.1±1.02% (n=4) of the peak I_{Na} at −20 mV, and it increased as V_{T} became more depolarised (FIG. 5D, right). In contrast, the time constant of the fast component (τ_{f}) in the presence of dmLSB was similar to that of the mono-exponential time constant under control conditions (FIG. 5D). These results indicate that dmLSB induced a slow component of inactivation at the expense of the normal fast component of I_{Na}.

### Example 9

### dmLSB has no Effect on the Steady-State Inactivation of I_{Na}

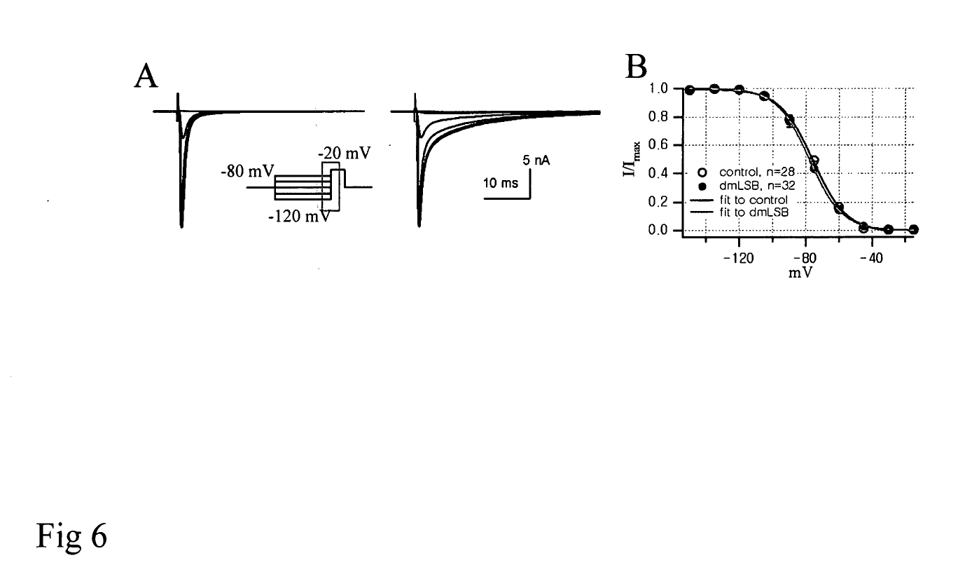

The effects of dmLSB on the steady-state inactivation of I_{Na} were studied using the pulse protocol shown in FIG. 6 (inset). Pre-pulses of 500 ms duration at variable potentials (from −150 to −15 mV in 15 mV steps) preceded a 50 ms test pulse to −20 mV. The superimposed current responses to the test pulse (−20 mV) are shown in FIG. 6A (left, control; right, 20 μM dmLSB). The slow inactivating I_{Na} in the presence of dmLSB indicates that dmLSB exerts its pharmacological effect (FIG. 6A, right). The peak amplitudes of I_{Na} are plotted against pre-pulse voltages in FIG. 6B. No differences in the steady-state inactivation curves were found before and after the dmLSB application, indicating that dmLSB has no effect on the steady-state inactivation of I_{Na}. The voltages for half inactivation were −75.7±0.61 mV (n=28) in control and −77.8±1.05 mV (n=32) in the presence of dmLSB (t-test, p>0.05).

### Example 10

### Dose-response Relationship

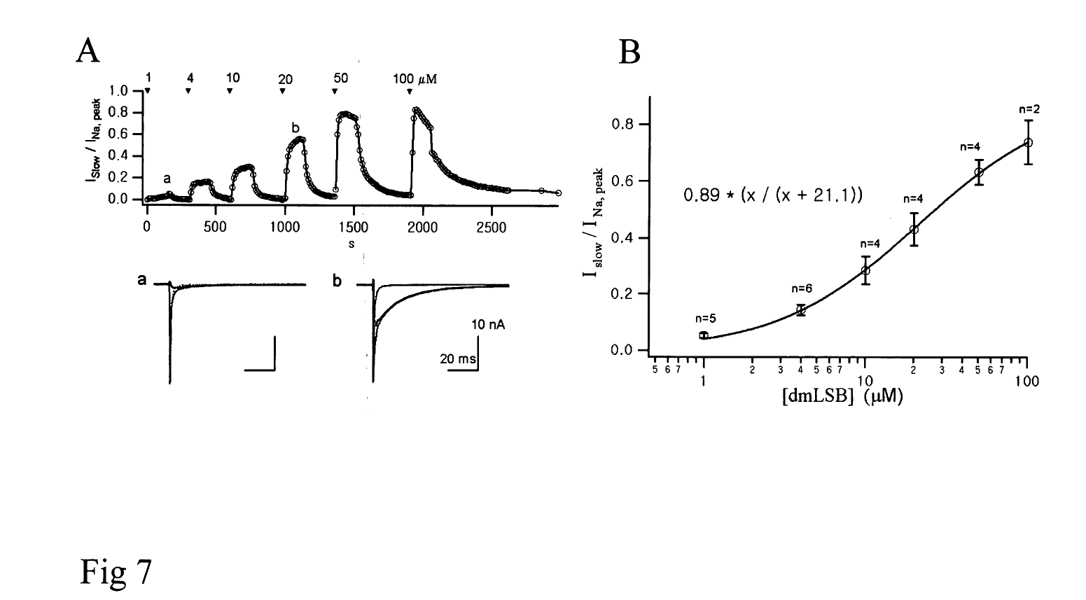

The effects of dmLSB on I_{Na} can be well demonstrated by subtracting the I_{Na} evoked by depolarisation to −20 mV under control conditions from that in the presence of dmLSB. Since dmLSB specifically affected the slow phase of inactivation, the difference current represented the slow inactivating I_{Na} (I_{Slow}) induced by dmLSB. To quantify the magnitude of the effect of dmLSB on I_{Na}, the relative amplitude of I_{Slow} to the peak I_{Na} was plotted. FIG. 7A shows the time course of change in the relative amplitude of I_{Slow} during the bath application of sequentially higher concentration of dmLSB.

The maximal effect of the drug at each concentration reached in ca. 150 seconds. Effects of dmLSB were reversible, although a longer time was required to wash out the drug effect at the higher concentration. From a set of similar experiments, dose-response relationship was obtained. To quantify the magnitude of the effect of dmLSB on I_{Na}, the relative amplitude of I_{Slow} to the peak I_{Na} was plotted as a function of dmLSB concentrations (FIG. 7B). The relationship was well fitted to the Hill equation with an EC₅₀ of 21.1 μM and a Hill coefficient of 1.02.

### Example 11

### Intracellular Application of dmLSB has no Effect on I_{Na} Inactivation

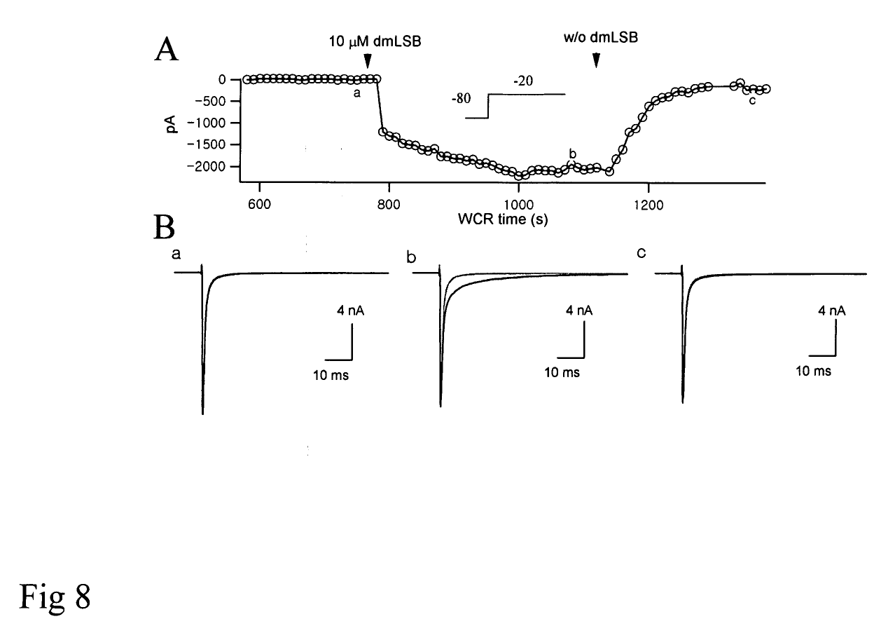

Since dmLSB is lipophilic, it is possible that it permeates the plasma membrane and binds to intracellular receptors or to Na channel protein itself. It is not clear whether its effects were mediated via a cell surface binding site, an intracellular site, or both. To test this possibility, a ventricular myocyte was perfused intracellularly with the patch pipette solution containing 20 μM dmLSB, and I_{Na} was monitored while patch pipette solution diffused-in. Meanwhile, the series resistance (R_{S}) was kept below 7 MΩ. As soon as the whole-cell patch was established, I_{Na} was recorded at a test potential of −20 mV, and this current trace was subtracted from subsequent I_{Na} records. The amplitudes of the subtracted currents were plotted as a function of whole-cell recording time (WCR time; FIG. 8A). Intracellular dmLSB exerted essentially no effect on I_{Na} (n=4), suggesting that the dmLSB binding site(s) is located on the surface of ventricular myocytes. No change in the inactivation kinetics of I_{Na} was observed until dmLSB was applied externally. When 5 μM dmLSB was bath-applied at ca 13 min of WCR time to the same cell, I_{Slow} appeared, indicative of the dmLSB effect.

### Example 12

### Effect of LSB Derivatives on I_{Na} Inactivation, APD and S-S Inactivation

Experimental methods for electrophysiology (recording currents and action potentials) are the same as described above for dmLSB.

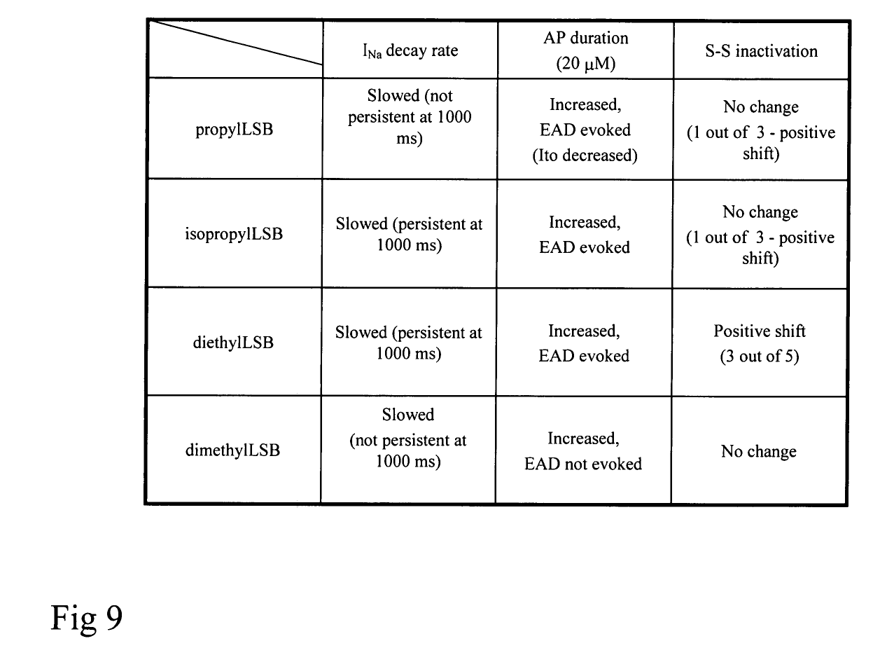

PropylLSB, isopropylLSB and diethylLSB were synthesized and tested for their effects on rat myocytes. PropylLSB displayed slowed I_{Na} inactivation, no significant effect on the V_{1/2}, no significant effect on late Na current, increased APD with EAD evoked and no change in steady state (s-s) inactivation. IsopropylLSB displayed slowed I_{Na} inactivation, no significant effect on the V_{1/2}, increased effect on late Na current, increased APD with EAD evoked and no change in steady state inactivation. DiethylLSB displayed slowed I_{Na} inactivation, positive shift effect of V_{1/2}, increased effect on late Na current, increased APD with EAD evoked and positive shift in steady state inactivation. The total summary is seen in FIG. 9. Similar to dmLSB, all of the tested derivatives have effect on the kinetics of Na current inactivation, causing slow inactivation. However, unlike dmLSB, slow inactivation was accompanied by persistent inward current, which is regarded to be arrythmogenic. Action potential experiments confirmed that these derivatives caused early afterdepolarization (EAD) which is regarded as an indication of arrythmogenic potential. Thereofore, propyl-, isopropyl-, and diethyl-form of LSB do not appear to be useful as a drug for the treating arrhythmia. Further, S-S inactivation represents voltage-dependent property of ion channel. FIG. 6 emplifies an experiment to obtain S-S inactivation curve, from which it was determined that dmLSB does not affect voltage-dependent property of Na channel inactivation, whereas it slows kinetics (speed) of inactivation.

### REFERENCES

CATERALL WA (1980). Neurotoxins that act on voltage-sensitive sodium channels in excitable membranes. Ann Rev Pharmacol Toxicol, 20, 15-43.

FUNG KP, WU J, ZENG LH, WONG HN, LEE CM, HON PM, CHANG HM & WU TW (1993). Lithospermic acid B as an antioxidant-based protector of cultured ventricular myocytes and aortic endothelial cells of rabbits. Life Sci, 53(12), PL189-93.

GRANT AO, CARBONI MP, NEPLIOUEVA V, STARMER CF, MEMMI M, NAPOLITANO C & PRIORI S (2002). Long QT syndrome, Brugada syndrome, and conduction system disease are linked to a single sodium channel mutation. J Clin Invest, 110(8), 1201-1209.

HAGIWARA N, IRISAWA H, KAMEYAMA M (1988). Contribution of two types of calcium currents to the pacemaker potentials of rabbit sin θ-atrial node cells. J Physiol. 395, 233-253.

KAMATA K, IIZUKA T, NAGAI M & KASUYA Y (1993). Endothelium-dependent vasodilator effects of the extract from Salviae Miltiorrhizae radix. A study on the identification of lithospermic acid B in the extracts. Gen Pharmacol, 24, 977-81.

KOHDA H, TAKEDA O, TANAKA S, YAMASAKI K, YAMASHITA A, KUROKAWA T, ISHIBASHI S (1989) Isolation of inhibitors of adenylate cyclase from dan-shen, the root of Salvia miltiorrhiza. Chem Pharm Bull (Tokyo). 37(5), 1287-90.

LEE GT, HA H, JUNG M, LI H, HONG SW, CHA BS, LEE HC & CHO YD (2003). Delayed treatment with lithospermate B attenuates experimental diabetic renal injury. J Am Soc Nephrol., 14, 709-20.

MACIANSKIENE R, BITO V, RAEYMAEKERS L, BRANDTS B, SIPIDO KR & MUBAGWA K (2003). Action potential changes associated with a slowed inactivation of cardiac gated sodium channels by KB130015. Br J Pharmacol, 139, 1469-1479.

MACIANSKIENE R, VIAPPIANI S, SIPIDO KR & MUBAGWA K (2003). Slowing of the inactivation of cardiac voltage-dependent sodium channels by the amiodarone derivative 2-Methyl-3-(3,5-diiodo-4-carboxymethoxybenzyl)benzofuran (KB 130015).

MULLER-EHMSEN J, BRIXIUS K & SCHWINGER RHG (1998). Positive inotropic effect of the novel Na+ channel modulator BDF 9198 in human failing and non-failing myocardium. J Cardio Pharma, 31, 684-689.

NAGAI M, NOGUCHI M, IIZUKA T, OTANI K & KAMATA K (1996). Vasodilator effects of des(alpha-carboxy-3,4-dihydroxyphenethyl)lithospermic acid (8-epiblechnic acid), a derivative of lithospermic acids in salviae miltiorrhizae radix. Biol Pharm Bull, 19, 228-232.

NARAHASHI T (1996). Neuronal ion channels as the target sites of insecticides. Pharmacology & Toxicology, 78, 1-14.

RAVEN U, AMOS GJ, EHRING T & HEUSCH G (1995). BDF 9148- A sodium channel modulator with positive inotropic action. Cardio Drug Rev, 3, 260-274.

RUEGG PC & NUESCH E (1995). The effect of a new inotropic agent, DPI 201-106 on systolic time intervals and the electrocardiogram in healthy subjects. Br J Clin Pharmacol, 24, 453-458.

SPENCER CI, YUILL KH, BORG JJ, HANCOX JC & KOZLOWSKI RZ (2001). Actions of pyrethroid insecticides on sodium currents, action potentials, and contractile rhythm in isolated mammalian ventricular myocytes and perfilsed hearts. The Journal of Pharmacology and Experimental Therapeutics, 298, 1067-1082.

TANAKA T, MORIMOTO S, NONAKA G, NISHIOKA I, YOKOZAWA T, CHUNG HY & URA H (1989). Magnesium and ammnium-potassium Lithospermates B, the active principles having a uremia-preventive effect from Salvia miltiorrhiza. Chem. Pharm. Bull., 37, 340-344.

VELDKAMP MW, VISWANATHAN PC, BEZZINA C, BAARTSCHEER A, WILDE AAM & BALSER JR (2000). Two Distinct Congenital Arrhythmias Evoked by a Multidysfunctional Nal Channel. Circ Res, 86, e91-e97.

WANG DW, MAKITA N, KITABATAK A, BALSER JR & GEORGE, A. (2000). Enhanced Na+ channel intermediate inactivation in Brugada syndrome. Circ Res, 87, e37- e43.

WANG SY & WANG GK (2003). Voltage-gated sodium channels as primary targets of diverse lipid-soluble neurotoxins. Cellular signalling, 15, 151-159.

YUILL KH, CONVERY MK, DOOLEY PC, DOGGRELL SA & HANCOX JC (2000). Effects of BDF 9198 on action potentials and ionic currents from guinea-pig isolated ventricular myocytes. Br J Pharmacol, 130, 1753-1766.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method of slowing inactivation of sodium current in a cell, which method comprises contacting the cell with an effective amount of dimethyl lithospermate B.

2. The method according to claim 1, wherein the dimethyl lithospermate B is administered by injection to a person.

3. The method according to claim 1, wherein the dimethyl lithospermate B is administered in the form of an herbal composition.

4. The method according to claim 3, wherein the herbal composition is obtained form an alcohol extract containing dimethyl lithospermate B.

5. The method according to claim 3, wherein the herbal composition comprises a plant of the genus Salvia.

6. The method according to claim 5, wherein the herbal composition comprises a plant Salvia miltiorrhiza.

7. The method according to claim 1, wherein the dimethyl lithospermate B is administered in the form of a drink.

8. The method according to claim 1, wherein the dimethyl lithospermate B is administered in the form of a tablet.

9. A method of treating a symptom of Brugada syndrome comprising administering to a person in need thereof a treatment effective amount of dimethyl lithospermate B.

10. The method according to claim 9, wherein the dimethyl lithospermate B is administered by injection to a person.

11. The method according to claim 9, wherein the dimethyl lithospermate B is administered in the form of an herbal composition.

12. The method according to claim 11, wherein the herbal composition is obtained form an alcohol extract containing dimethyl lithospermate B.

13. The method according to claim 11, wherein the herbal composition comprises a plant of the genus Salvia.

14. The method according to claim 13, wherein the herbal composition comprises a plant Salvia miltiorrhiza.

15. The method according to claim 9, wherein the dimethyl lithospermate B is administered in the form of a drink.

16. The method according to claim 9, wherein the dimethyl lithospermate B is administered in the form of a tablet.

17. A method of treating arrhythmia comprising administering to a person in need thereof a treatment effective amount of dimethyl lithospermate B.

18. The method according to claim 17, wherein the dimethyl lithospermate B is administered by injection to a person.

19. The method according to claim 17, wherein the dimethyl lithospermate B is administered in the form of an herbal composition.

20. The method according to claim 17, wherein the dimethyl lithospermate B is administered in the form of a drink.
